(19) United States
(12) Patent Application Publication
(10) Pub. No.: US 2004/0241316 A1
(43) Pub. Date: Dec. 2, 2004

(54) Radioactively coated stents

(76) Inventors: Ludger Dinkelborg, Berlin (DE); Peter Muschick, Ladeburg (DE); Bernard Noll, Freital (DE); Heidemarie Goerner, Dresden (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/471,232

(22) PCT Filed: Mar. 7, 2002
(86) PCT No.: PCT/EP02/02531

(30) Foreign Application Priority Data
Mar. 9, 2001 (DE) 10112518.6

(51) Int. Cl. B05D 1/18; A61F 2/06

(52) U.S. Cl. 427/2.25; 623/1.15; 623/1.46; 600/431

(57) ABSTRACT

The invention relates to novel radioactive stents and to methods for the production thereof.

The invention relates to new radioactively coated stents as well as processes for their production.

Radioactively coated stents as well as processes for their production were already described in Patents DE 197 24 223, DE 197 24 229, DE 197 24 230 as well as in Patent Application WO 98/48851. The stents described there are used as vascular implants for preventing restenoses.

An undesirable side effect after stent implantation is that frequently a restenosis, which is referred to among experts as a “candy wrapper” effect (Albiero et al. Edge Restenosis After Implantation of High Activity P-32 Radioactive beta-Emitting Stents, Circulation 2000, 101: 2454-2457), is observed at the stent ends after a certain time. This means that inside the stent, the restentosis is prevented; however, after a certain time, a restenosis of the blood vessel occurs at the stent ends. To date, no stents are known that reliably prevent restenosis not only inside the stent but also at its outer ends.

The object of this invention is therefore to provide new, improved radioactive stents, after whose implantation the “candy wrapper” effect no longer occurs and that reliably prevent restenosis both within the stent and at its ends. Another object of this invention is to provide new, improved processes that are simple to implement for the production of radioactive stents.

These objects are achieved by the new radioactive stents and the processes for their production, as they are defined in the claims.

It has been found, surprisingly enough, that stents that are coated by radioactive isotopes and that release β-radiation within a radius of at least 10 mm and with a maximum energy of greater than 2 MeV, do not show any “candy wrapper” effects in animal experiments and reliably prevent restenosis both inside the stent and at its ends. A suitable isotope is, e.g., Re-188, which has a radius of 11 mm and an energy of 2.11 MeV. The short half-life of under 91 hours, in the case of Re-188 about 17 hours, also contributes to the fact that the restenosis is not induced at the stent ends.

The production of the radioactive stent is carried out according to new, improved processes.

In the known processes for the production of radioactive stents, a non-radioactive stent is immersed in a solution that contains the radioactive isotope in ionic form, and the isotope is then chemically deposited on the stent.

It has been found, surprisingly enough, that the radioactive stents have improved properties if they undergo a sintering process under high vacuum at 600 to 1100° C. after the radioactive metal is deposited. The radioactivity that is first applied in a metastable manner is fixed by this sintering process, such that the stent can be better managed, and the activity is not lost after implantation. The deposited layer is thus fixed in a mechanically stable manner, such that the radioactivity can no longer be removed just by washing in water or ethanol. In addition, the appearance of the surface of the stent is improved from matt-black to matt-metallic, which is an indication of a smoother surface.

In the new, improved process, a non-radioactive stent is first immersed in a solution that contains the radioactive isotope, preferably Re-188. The perrhenate is preferably provided in physiological common salt solution. Then, hydrochloric acid (HCl) is added to the solution, such that as a result, the labeling solution contains 1 M HCl. The reaction vessel is then tightly sealed and heated for some minutes to about 100° C. After cooling, the stent is removed and heated in another processing step under high vacuum (<10⁻³ Torr) to 600 to 1100° C., preferably to about 950° C.

Baking under high vacuum (<10⁻³ Torr) at about 950° C. results in considerably improved surface conditions of the stents. If the baking step takes place at normal pressure (760 Torr), then a large portion of the activity (60-80%) is already lost during the baking. The surface of the stents is visibly damaged. If the heating takes place in inert-gas atmosphere (argon, nitrogen, helium), the thus treated stents after incubation in physiological common salt solution then show reddish-brown coatings, and the incubation solutions show reddish-brown precipitates.

The electron-microscopic imaging (500× magnification) of the surfaces of the stents that are labeled according to the improved process still show clear grain boundaries, which are comparable, for example, to those of the untreated stents. To the naked eye, the surfaces seem somewhat duller.

With an alternative production process, first a non-radioactive stent is immersed in a solution that contains the radioactive isotope, preferably Re-188. The perrhenate is preferably provided in physiological common salt solution. Instead of hydrochloric acid, other acids, such as, e.g., sulfur acid, are used in this alternative process. In this case, the subsequent heating step in a vacuum can be eliminated. A washing step, e.g., in dilute or undiluted ethanol, as well as a sterilization process, e.g., in a drying oven or sterilizer, can then follow. The sterilization process is performed at 180 to 220° C., preferably at about 200° C. In this alternative production process, the baking step under high vacuum is unnecessary.

In the stents that are labeled in sulfuric-acid solution, no deposited coating can be detected under the microscope (16× magnification); the surface has a silvery luster and only the grain boundaries stand out as different in comparison to the untreated stent. No significant change of the surface occurs by the sterilization process.

As non-radioactive stents, commercially available stents, such as, e.g., the Wall stent, the Palmaz stent or the Palmaz-Schatz stent, the Wiktor stent, the AVE stent, the GFX stent, the multilink stent, the radius stent, the NIR stent, the Jomed stent, the Angiomed stent, the Nitinol stent and other stents, are used. Metal stents are preferred.

With the thus produced stents, incubation tests were performed in human blood or in physiological common salt solution. Stents that were labeled under the optimized conditions still contain more than 90% of the applied radioactivity after 66 hours of incubation in human whole blood or physiological common salt solution at 37° C.

The analysis of the chemical composition of the stent surfaces indicated that the labeling step with a concentration of the elements molybdenum, silicon and rhenium (measured in ¹⁸⁶Re-labled stents) is connected to the stent surfaces that are essential components of the stent high-grade steel.

Parts of the non-radioactive stent can be coated by paint before immersion in the solution that contains the radioactive isotope. It was possible to show that only a little or no radioactivity is measured on the thus coated spots after the sintering process. Traces of paint are no longer present on the stent after the sintering process. It thus is possible to structure the activity of the stents regionally differently and individually. Stents that have more or less activity in comparison to the residual stent surfaces on the ends thus can be produced (“cold end” or “hot end” stents).

In a pig study with 16 pigs were implanted a) two Re-186-coated coronary, 16 mm stents in the Ramus interventricularis anterior (RIVA) and Ramus circumflexus (RCX) of the left coronary artery, and b) two Re-186-coated peripheral, 58 mm stents.

In the follow-up tests (angiography and ultrasound), in the coronary stents (group a) in contrast to the control, it was found that Re-186-coated stents on both stent ends developed new stenotic lesions (“edge or candy wrapper” effects), while the primary vascular segment to be treated in the stent itself (target lesion) was free of a restenosis.

Surprisingly enough, in another pig study, which was performed with stents of the same dimensions, which were coated by Re-188 instead of by Re-186, however, it was found in the follow-up test that the Re-188-coated stents reliably prevent both restenosis at the stent ends and in the stent itself both in the coronary vessels and in the peripheral vessels (groups a) and b)). The inventive object was thus achieved.

In the following examples, the production of radioactive stents as well as their activity in vivo is described in detail.

### EXAMPLES

### Example 1

A Palmaz stent (20 mm, Johnson & Johnson) or a coronary Wave stent (16 mm, JoMed) is covered with 1.5 ml of labeling solution, consisting of x ml of [¹⁸⁶Re] or [¹⁸⁸Re] sodium perrhenate solution, x ml of 2 M HCl and y ml of 1 M HCl (2x+y=1.5 ml). The reaction vessel is tightly sealed and heated for 10-15 minutes at 100° C. Then, the reaction vessel is cooled to room temperature in an ice bath, the stent is removed, and rinsed in ethanol to remove adhering labeling solution and easily removable radioactivity. About 20% of the initially deposited activity, whereby the latter is, depending on the labeling period, about 40-60% of the [¹⁸⁸Re]/[¹⁸⁸Re] sodium perrhenate that is introduced into the labeling solution, is dissolved in this case. To fix this activity that is applied in a metastable manner, the stents must then be converted into a quartz ampoule and heated under high vacuum (<10⁻³ Torr) for 15 minutes at 950° C. During heating, about 2-5% of the radioactivity is removed from the stent. The total labeling yield is about 30-45% depending on the labeling period.

### Example 2

A Palmaz stent (58 mm, Johnson & Johnson) or a peripheral Wave stent (58 mm, JoMed) is covered with 4.5 ml of labeling solution, consisting of x ml of [¹⁸⁶Re] or [¹⁸⁸Re] sodium perrhenate solution, x ml of 2 M HCl and y ml of 1 M HCl (2x+y=4.5 ml). The reaction vessel is tightly sealed and heated for 10-15 minutes at 100° C. Then, the reaction vessel is cooled to room temperature in an ice bath, the stent is removed and rinsed in ethanol to remove adhering labeling solution and easily removable radioactivity. About 20% of the initially deposited activity, whereby the latter is, depending on the labeling period, about 40-60% of the [¹⁸⁸Re]/[¹⁸⁸Re] sodium perrhenate that is introduced into the labeling solution, is dissolved in this case. To fix this activity that is applied in a metastable manner, the stents must then be converted into a quartz ampoule and heated under high vacuum (<10⁻³ Torr) for 15 minutes at 950° C. During heating, about 2-5% of the radioactivity is removed from the stent. The total labeling yield is about 30-45% depending on the labeling period.

### Example 3

A Palmaz stent (20 mm, Johnson & Johnson) or a coronary Wave stent (16 mm, JoMed) is immersed on both ends in a paint (Zapon paint) and provided with a paint layer that is 2-4 mm in each case. The paint is dried in the air or in a hot stream of air.

The thus treated stent is covered with 1.5 ml of labeling solution, consisting of x ml of [¹⁸⁶Re] or [¹⁸⁸Re] sodium perrhenate solution, x ml of 2 M HCl and y ml of 1 M HCl (2x+y=1.5 ml). The reaction vessel is tightly sealed and heated for 10-15 minutes at 100° C. Then, the reaction vessel is cooled to room temperature in an ice bath, the stent is removed and rinsed in acetone to remove the paint at the stent ends, adhering labeling solution and easily removable radioactivity. About 20% of the initially deposited activity, whereby the latter is, depending on the labeling period, about 40-60% of the [¹⁸⁸Re]/[¹⁸⁸Re] sodium perrhenate that is introduced into the labeling solution, is dissolved in this case. To fix this activity that is applied in a metastable manner, the stent is then converted into a quartz ampoule and heated under high vacuum (<10⁻³ Torr) for 15 minutes at 950° C. During heating, about 2-5% of the radioactivity is removed from the stent. The total labeling yield is about 30-45% depending on the labeling period.

### Example 4

A Palmaz stent (58 mm, Johnson & Johnson) or a peripheral Wave stent (58 mm, JoMed) is immersed on both ends in a paint (Zapon paint) and provided with a paint layer, in each case 2-6 mm. The paint is dried in the air or in a hot stream of air, covered with 4.5 ml of labeling solution, consisting of x ml of [¹⁸⁶Re] or [¹⁸⁸Re] sodium perrhenate solution, x ml of 2 M HCl and y ml of 1 M HCl (2x+y=4.5 ml). The reaction vessel is tightly sealed and heated for 10-15 minutes at 100° C. Then, the reaction vessel is cooled to room temperature in an ice bath, the stent is removed and rinsed in acetone to remove paint at the stent ends, adhering labeling solution and easily removable radioactivity. About 20% of the initially deposited activity, whereby the latter is, depending on the labeling period, about 40-60% of the [¹⁸⁸Re]/[¹⁸⁸Re] sodium perrhenate that is introduced into the labeling solution, is dissolved in this case. To fix this activity that is applied in a metastable manner, the stents must then be converted into a quartz ampoule and heated under high vacuum (<10⁻³ Torr) for 15 minutes at 950° C. During heating, about 2-5% of the radioactivity is removed from the stent. The total labeling yield is about 30-45% depending on the labeling period.

### Example 5

A Palmaz stent (20 mm, Johnson & Johnson) or a coronary Wave stent (16 mm, JoMed) is covered with 1.5 ml of labeling solution (sterile Re-generator eluate in physiological common salt solution, consisting of x ml of [¹⁸⁶Re] or [¹⁸⁸Re] sodium perrhenate solution, x ml of 2 M H₂SO₄ and y ml of 1 M H₂SO₄ (2x+y=1.5 ml). The reaction vessel is tightly sealed and heated for 10-15 minutes at 100° C. Then, the reaction vessel is cooled to room temperature in an ice bath. The stent is removed and placed in a reaction vessel that contains 2 ml of aqueous ethanol (e.g., 50%). Optionally, in an ultrasound bath, the stent is rinsed to remove easily removable radioactivity. About 30-50% of the initially deposited activity is dissolved in this case. Depending on the labeling period, the labeling yield is about 10% of the [¹⁸⁸Re]/[¹⁸⁸Re] sodium perrhenate that is introduced into the labeling solution. The stent is then heated in a drying oven/sterilizer to about 200° C. and then is present in sterilized form.

### Example 6

A Palmaz stent (58 mm, Johnson & Johnson) or a peripheral Wave stent (58 mm, JoMed) is covered with 4.5 ml of labeling solution, consisting of x ml of [¹⁸⁶Re] or [¹⁸⁸Re] sodium perrhenate solution, x ml of 2 M H₂SO₄ and y ml of 1 M H₂SO₄ (2x+y=4.5 ml). The reaction vessel is tightly sealed and heated for 10-15 minutes at 100° C. Then, the reaction vessel is cooled to room temperature in an ice bath.

The stent is removed and introduced into a reaction vessel that contains 2 ml of aqueous ethanol (about 50%). In an ultrasound bath, the stent is rinsed to remove easily removable radioactivity. About 30-50% of the initially deposited activity is dissolved in this case. Depending on the labeling period, the labeling yield is about 10% of the [¹⁸⁸Re]/[¹⁸⁸Re] sodium perrhenate that is introduced into the labeling solution. The stent is then heated in a drying oven/sterilizer to about 200° C. and then is present in sterilized form.

### Example 7

Re-186-Labeled Stents in the Animal Model of the Pig

Animals: Gottingen mini-pigs, male, about 30 kg, n=17

Anesthesia: Introduction of anesthesia: ketamine 10 mg/kg, stresnil 4 mg/kg, ketamine 10 mg/kg i.m. and ketamine, 200 mg, valium 5 mg i.v. Anesthesia during the OP: fentanyl/droperidol and nitrous oxide/oxygen 3:1 (0.8/2.4 ml) with enfluranes 1.5-2%

Premedication: 1 day before the OP, 500 mg of aspirin p.o. and 300 mg of clopidrogel p.o.

Medication: On the day of the OP: 5 ml of aspisol (500 mg) i.v., 1 ml (5000 IE) of liquemin i.a., 3 ml (150 μg) of nitroglycerin i.a., 1.7 ml of tardomyocel i.m.

Subsequently, up to the time of sacrifice: 1× daily 75 mg of clopidrogel and 100 mg of ASS p.o.

Stents: a) 16 mm Coronary Jomed stents that are dilated to 3.5 mm, coated by Re-186 according to the above-described process with a sintering process:

For RIVA: 10.9±1.58 MBq (n=9)

For RCX: 18.1±1.6 MBq (n=8)

b) 58 mm Peripheral JoMed stents that are dilated to 7 mm, coated by Re-186 according to the above-described process with a sintering process:

For left carotids: 77.5±10.4 MBq (n=9)

For right carotids: 136.8±4.7 MBq (n=8)

c) Untreated coronary RIVA (n=8)/RCX(n=8) and peripheral (n=12) stents as a control

Execution of the Test:

The animals are anesthetized with an i.m. anesthesia. Then, venous access is made in an ear vein, and the anesthesia is intravenously introduced via this access.

The tube (Gr. 10, Rüsch Company) is wetted with a lubricant (Meaverin® gel) and inserted in the trachea with the aid of a tongue depressor (laryngoscope) and fixed with the bulb of the tube. The inner thighs of the hind legs are shaved, the animal is covered with a sterile OP cloth, and the shaved spots are disinfected with an iodine-containing tincture (Braunol 2000).

With the aid of a puncture needle, under ultrasonic monitoring, the femoral artery is punctured.

Via a guide wire, the lock (Cordis F8-Avanti Einführbesteck Company) is inserted and fixed. To prevent the formation of blood clots, 100 IE/kg of liquemin is administered intraarterially.

Via the lock, a guide catheter is advanced to be able to advance the balloon catheter undamaged.

Under x-ray monitoring, the right and left carotids are damaged by means of balloon catheter Gr. 8 by pressing to 8 bar three times. Then, the stents are placed with the aid of a balloon catheter Gr. 7, and the catheter is pressed to 8 bar for 30 seconds. Then, a control angiography is carried out.

In each case, the coronary stents are placed without a preliminary dilation of the vessels in the Ramus circumflexus (RCX) and Ramus intraventricularis anterior (RIVA) by means of a balloon catheter Gr. 3 and dilation to 10 bar for 30 seconds under x-ray monitoring. Then, a control angiography is carried out. In the work on coronaries, nitroglycerin is given as required when spasms occur. After the end of the test, the lock is drawn, and a pressure bandage is applied. 500 mg of aspisol and 1.7 ml of tardomyocel are administered intramuscularly.

Until the animal breathes on its own spontaneously, artificial respiration with ambient air is administered to the animal. Then, the tube is drawn.

The stability of the Re-186 coating is examined by means of gamma camera (Elscint SP-4 HR, energy: 63 keV/136 keV) after the OP end and 2 hours post stent administration. The animals are kept under anesthesia for this time with ketamine i.v., if necessary.

The awakening of the animals is carried out under observation in several cages with food and water.

Follow-Up Test: 8 Weeks After Stent Implantation

The animals are anesthetized with an i.m. anesthesia. Then, venous access is made in an ear vein, and the anesthesia is intravenously introduced via this access.

The tube (Gr. 10, Rüsch Company) is wetted with a lubricant (Meaverine gel) and inserted in the trachea with the aid of a tongue depressor (laryngoscope) and fixed with the bulb of the tube.

The inner thighs of the hind legs are shaved, the animal is covered with a sterile OP cloth, and the shaved spots are disinfected with an iodine-containing tincture.

With the aid of a puncture needle and under ultrasonic monitoring, the femoral artery is punctured.

Via a guide wire, the lock (Cordis F8-Avanti Einführbesteck Company) is inserted and fixed.

To prevent the formation of blood clots, 100 IE/kg of liquemin is administered intraarterially.

A guide catheter is advanced into the stented vessels, and the vessels are visualized by means of angiography (Ultravist-370, Schering Company) and IVUS ultrasonic study. In the study of coronary vessels, nitroglycerin is administered, if necessary, when spasms occur.

After the end of the test, the lock is drawn, and a pressure bandage is applied. Until the animal breathes on its own spontaneously, artificial respiration with ambient air is administered to the animal. Then, the tube is drawn.

The awakening of the animals is carried out under observation in several cages with food and water.

Stent Removal: 6 Months after Stent Implantation

The animals are anesthetized by means of an i.m. anesthesia. Then, venous access is made in an ear vein, and the anesthesia is intravenously introduced via this access.

In addition, Narcoren (4 ml) and fentanyldroperidol (0.1/5 mg) are administered i.v.

To prevent the formation of blood clots, 1000 IE of liquemin is administered i.v. The animals are sacrificed with 20 ml of KCl (7.45%) i.v.

Then, the removal of vascular pieces that are rinsed and fixed with formalin-containing fixing solution (2% paraformaldehyde solution in cacodylate buffer) is carried out.

Then, histological studies of the vessels in the stent area and their ratings are performed.

Results:

The in-stent restenosis was significantly suppressed both in the two areas of the left coronary as well as in the carotids. As representative of any actual animal, marginal effects (candy wrapper) can be observed in all vascular origins examined at the dosages of Re-186 that are used.

### Example 8

Pig Study of Re-186 Cold-End Stents and Low Dosages

Animals: Gottingen mini-pigs, male, about 30 kg, n=4 (valid for Examples 8 and 9).

Anesthesia: Fentanyl/droperidol and nitrous oxide/oxygen 3:1 (0.8/2.4 ml) with enfluranes 1.5-2%

Premedication: 1 day before the OP, 500 mg of aspirin p.o. and 300 mg of clopidrogel p.o.

Medication: On the day of the OP: 5 ml of aspisol (500 mg) i.v., 1 ml (5000 IE) of liquemin i.a., 3 ml (150 μg) of nitroglycerin, 1.7 ml of tardomyocel Subsequently, up to the time of sacrifice: 1× daily of 75 mg of clopidrogel and 100 mg of ASS p.o.

Stents: a) 16 mm coronary Jomed stents that are dilated to 3.5 mm, coated with a sintering process according to the above-described process:

RE-186 complete: 0.8/1.9 MBq (RCX/RIVA)

b) 58 mm Peripheral Jomed stents that are dilated to 7 mm, coated by a sintering process according to the above-described process:

RE-186 cold end: 12/27 MBq (re A.c./re A.c.)*

Complete: 11/28 MBq (li A.c./li A.c.)*

*right common carotid artery (re A.c.), left common carotid artery (li A.c.)

c) The control (n=1, li A.c.) is coated by cold rhenium (process as described above, with a sintering process) (valid for Examples 8 and 9).

On each side, cold-end stents have a 4 mm piece without activity (for production, see Examples 3 and 4).

Execution: See Example 7

Follow-Up Test: 4 weeks after stent implantation

See Example 7

Result:

Also in the case of these reduced Re-186 dosages, in contrast to the controls, no in-stent restenosis could be observed both in the peripheral arteries as well as in the coronary arteries. In the carotids, also no side effects could be observed. In the left coronary artery, however, marginal effects also occurred in the case of this reduced dosage. In the case of the “cold-end stents,” marginal effects were observed in the area of the stent ends. The stents that were labeled with the described process, which were used without radioactivity, do not show any increased formation of restenosis in comparison to the control (see Example 7).

### Example 9

Re-188 Pig Study

Animals: Göttingen mini-pigs, male, about 30 kg, n=4 (valid for Examples 8 and 9).

Anesthesia: Fentanyl/droperidol and nitrous oxide/oxygen 3:1 (0.8/2.4 ml) with enfluranes 1.5-2%

Premedication: 1 day before the OP, 500 mg of aspirin p.o. and 300 mg of clopidrogel p.o.

Medication: On the day of the OP: 5 ml of aspisol (500 mg) i.v., 1 ml (5000 IE) of liquemin i.a., 3 ml (150 μg) of nitroglycerin, 1.7 ml of tardomyocel

Subsequently, up to the time of sacrifice: 1× daily of 75 mg of clopidrogel and 100 mg of ASS p.o.

Stents: a) 16 mm coronary Jomed stents that are dilated to 3.5 mm, coated by a sintering process according to the above-described process:

Re-188 cold end: 0.8/1.7 MBq (RIVA/RIVA)

Complete: 1.5/3 MBq (RCX/RCX)

b) 58 mm Peripheral Jomed stents that are dilated to 7 mm, coated by a sintering process according to the above-described process:

Re-188 cold end: 18 MBq ((li A.c.)

Complete: 21 MBq (re A.c.)

c) the control (n=1, li A.c.) is coated by cold rhenium (valid for Examples 8 and 9).

On each side, cold-end stents have a 4 mm piece without activity.

Execution: See Example 7

Follow-Up Test: 4 weeks after stent implantation, see Example 7

Result:

In all Re-188-labeled stents examined, neither an in-restenosis nor marginal effects can be observed.

1. Process for the production of radioactive stents that release β-radiation within a radius of at least 10 mm and with an energy of greater than 2 MeV, in which a non-radioactive stent is immersed in a solution that contains the radioactive isotope in ionic form, and the isotope is then chemically deposited on the stent, characterized in that the radioactive stent then is subjected to a sintering process under high vacuum, which is <10⁻³ Torr, at 600 to 1100° C.

2. Process according to claim 1, wherein the sintering process is performed at 950° C.

3. Process according to claim 1, wherein the radioactive isotope has a half-life of less than 91 hours.

4. Process according to claim 1, wherein the radioactive isotope has a half-life of 17 hours or less.

5. Process according to one of claims 1-4, wherein the radioactive isotope is Re-188.

6. Process for the production of radioactive stents, in which a non-radioactive stent is immersed in a solution that contains the radioactive isotope in ionic form, and the isotope is then chemically deposited on the stent, wherein the solution contains an acid, especially sulfuric acid or hydrochloric acid.

7. Process according to claim 6, wherein the solution contains additional common salt.

8. Process according to one of claims 6 or 7, wherein the stent is heated in a further processing step in a drying oven or a sterilizer to 180 to 220° C.

9. Process according to one of claims 6 to 8, wherein the radioactive isotope releases β-radiation within a radius of at least 10 m and with an energy of greater than 2 MeV.

10. Process according to one of claims 6 to 9, wherein the radioactive isotope has a half-life of less than 91 hours.

11. Process according to one of claims 6 to 9, wherein the radioactive isotope has a half-life of 17 hours or less.

12. Process according to one of claims 6 to 9, wherein the radioactive isotope is Re-188.

13. Radioactive stents that can be produced according to a process according to one of claims 1 to 12.

14. Radioactive stents according to claim 13, wherein the stent ends have a different radioactivity from that of the middle of the stent.

15. Use of Re-188 for coating stents according to claim 13 or 14 that prevent restenosis of a blood vessel within the stent and also on the stent ends.
